(19) United States
(12) Patent Application Publication
(10) Pub. No.: US 2004/0260315 A1
(43) Pub. Date: Dec. 23, 2004

(54) Expandable tissue support member and method of forming the support member

(76) Inventors: Jeffrey R. Dell, Knoxville, TN (US); Michele Gandy Davis, Forsyth, GA (US); Gary Teague, Conyers, GA (US)

Correspondence Address: Matthew W. Siegal, Strook & Strook & Lavan LLP, 180 Maiden Lane, New York, NY, 10038, US

(21) Appl. No.: 10/463,017

(22) Filed: Jun. 17, 2003

(51) Int. Cl. A61F 2/02

(52) U.S. Cl. 606/151; 623/23.72

(57) ABSTRACT

An implant member has a body made from biocompatible material, and this body has slits formed therein. The slits open when the body is subjected to tension. The implant member is made by providing a body member and forming slits in the body. The slits are dimensioned and disposed so that the slits open when force is applied to the body.

### BACKGROUND OF THE INVENTION

Various surgical techniques benefit from the use of non-native flat supporting members to provide the patient's own tissue with additional mechanical strength. Such supporting members can be made from synthetic material, natural material, whether harvested from the patient or elsewhere, or composites of both synthetic and natural materials. When using harvested natural material, it may be desirable to treat the source tissue to alter its physical properties to insure it is biocompatible and does not cause an adverse reaction with the patient's immune system.

One example of a sheet-like support structure for use in a range of surgical techniques is described in U.S. Pat. No. 6,197,036. This patent discloses a pelvic floor reconstruction surgical patch made from natural or synthetic biocompatible material. According to the '036 patent, the preferred material for use in the patch is synthetic fabric made from polyester, more preferably, collagen coated polyester. The patch has a number of holes which are arranged in a specific manner with respect to the patch's corners.

Patches for use in surgical procedures can be made from synthetic mesh material, for example, polypropylene. Although easy to sterilize and inexpensive, synthetic mesh material has a number of shortcomings. Perhaps most important, when synthetic mesh material is used as a support member, the roughness of the synthetic mesh may lead to abrasion of the patient's tissue, and that can cause infection and/or erosion of the tissue.

Another material that can be used as a patch to reinforce soft tissue is processed porcine intestinal tissue. Examples of support structures made from such material include the Surgisis® Gold™ Hernia Repair Grafts, the Surgisis® Soft Tissue Grafts, and the Surgisis® IHM™ Inguinal Hernia Matrix, all manufactured by Cook Surgical, of Bloomington, Ind. and described in Cook Surgical's literature.

Another article of interest is the Stratasis® TF sling support, suitable for use in urethral sling suspension procedures for treating female incontinence, manufactured by Cook Urological, Inc. of Spencer, Ind. The Stratasis® TF support is a three-dimensional extracellular matrix which includes collagen, non-collagenous proteins, and biomolecules that is made of natural biomaterial derived from the small intestine of pigs. When implanted, the Stratasis® TF support is gradually replaced by the patient's body.

Although natural support members offer many benefits, for example, they are not abrasive, they also are generally more expensive than their synthetic counterparts, since such support members are derived from natural source materials that must be treated to insure sterility, stability and biocompatibility.

Given the expense of natural support members, it is desirable to reduce the amount of natural material used in each support member without also reducing the strength or durability of that support member.

There also exists a long-felt and unsolved need for a support system which offers the respective cost and tolerance benefits of both synthetic and natural materials, without the drawbacks of either of those articles.

### SUMMARY OF THE INVENTION

First, it should be understood that although this disclosure speaks in part of rectocele procedures, this invention is not to be limited thereto. By way of non-limiting example, the devices and techniques taught herein could be employed to support body organs such as the bowel or bladder. Consequently, all portions of this description should be understood to encompass such alternative uses of this invention.

By using this invention one can obtain an implant member offering reduced wound dehiscence and a greater ability to conform to the tissue in the area of the implant site. For example, this implant member can be used at a area that is trapezoidal.

This invention also can reduce the amount of natural material required to fabricate an implant member of given size.

One aspect of this invention is an implant member that has a body made from biocompatible material. The body has slits formed therein, and these slits open when the body is subjected to tension.

Yet another aspect of this invention is a method of manufacturing an implant member by providing a body member and forming slits in the body. The slits are dimensioned and disposed so that the slits open when force is applied to the body.

One benefit of this invention is that it reduces material expenses by allowing a small piece of biocompatible implant material to be used to cover a larger area. Furthermore, the resulting processed material is more pliable and soft. The processed material can conform around irregular surfaces and anatomical structures. This processed material, owing to its slit structure, also can expand in response to changes in the force applied thereto that may occur as the patient moves about, or as internal body structures move, and this will increase patient comfort.

### BRIEF DESCRIPTION OF THE DRAWINGS

In the drawing figures, which are merely illustrative, and wherein like reference characters denote similar elements throughout the several views:

FIG. 1 is a perspective view of a support member prepared in accordance with this invention shown in the relaxed (unexpanded) state;

FIG. 2 is a perspective view of the support member under tension and shown in the expanded state; and

FIGS. 3A and 3B depict a support in accordance with this invention in the unexpanded and expanded state, respectively;

FIG. 4 depicts another support member in accordance with this invention;

FIGS. 5 and 6 depict a further support member in accordance with this invention in the relaxed and tensioned states, respectively; and

FIGS. 7 and 8 depict still another support member in accordance with this invention in the relaxed and tensioned states, respectively.

### DETAILED DESCRIPTION OF THE PREFERRED EMBODIMENTS

Referring now to the drawings, the various embodiments of the present invention will be discussed in detail.

Among the materials which can serve as support members for implantation in the body is acellular dermal tissue and, more specifically, porcine dermal tissue. Such dermal tissue material must, however, be processed to render it biocompatible. One scheme for preparing biocompatible porcine dermal tissue is set forth in U.S. Pat. No. 5,397,353 to Oliver et al. and owned by Tissue Science Laboratories plc. one presently-preferred material that can be used in the implant strip 15 is Pelvicol™ implant material, distributed by C.R. Bard, Inc. of Murray Hill, N.J. and produced by Tissue Science Laboratories PLC, of Aldershot, Hampshire, United Kingdom. The material described in the '353 patent is particularly preferable for use in the present invention because such material is non-antigenic and is recolonized and revascularized by the host tissue. Also, owing to cross-linking, this material is non-resorbable, meaning it is not processed and eventually absorbed by the patient's body. Consequently, an implant made from this material will provide permanent support. In contrast to a procedure using a support made from resorbable material, the patient will not have to undergo later surgery to replace the support. It should be understood that other types of dermal tissue also could be used.

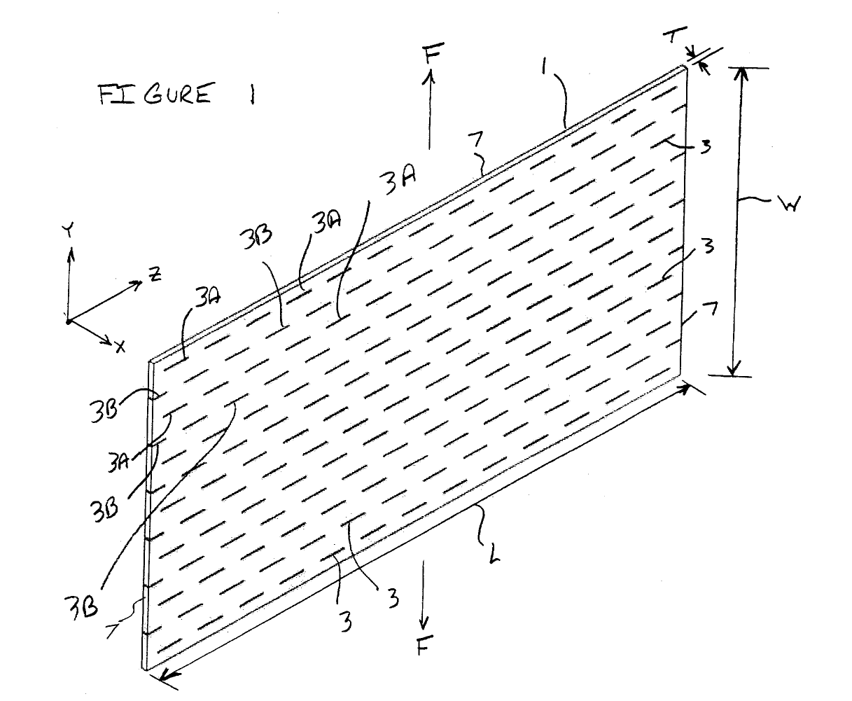

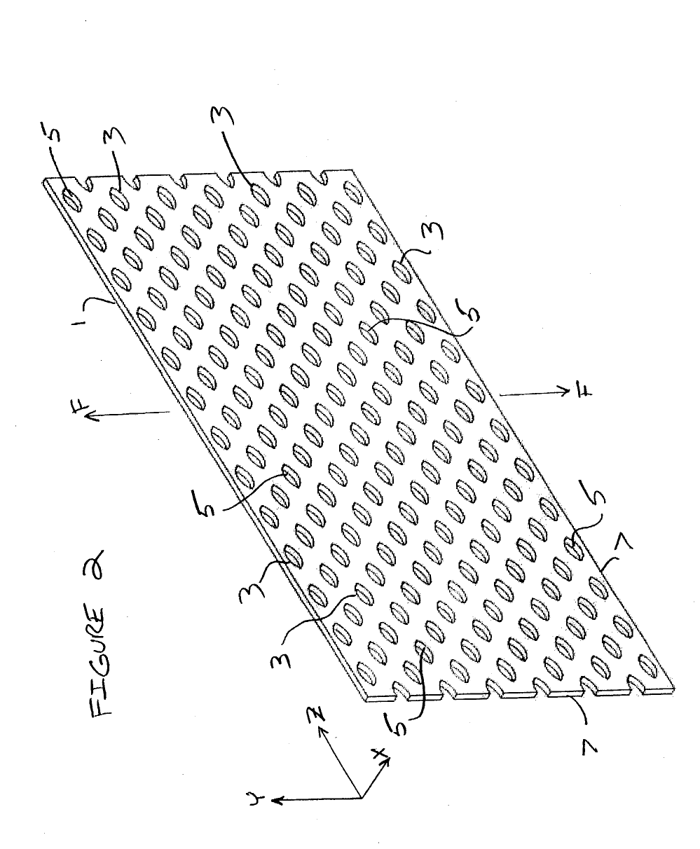

FIGS. 1 and 2 depict a rectangular implant member 1 prepared in accordance with this invention. As depicted in FIG. 1, the present invention is directed to an implant member 1 having a number of slits 3 formed therein. Implant member 1 can be a flat piece of biocompatible material, and, more preferably, is acellular dermal tissue prepared in accordance with the '353 patent, most preferably, porcine. Such materials is preferably rectangular, although other shapes such as square and round could be used, depending upon the particular type of surgery that is being performed and the shape of the body tissue that is being repaired.

Implant member 1 could be used for the surgical repair of damaged or ruptured soft tissue membranes, and, more specifically, for the repair of scrotal hernias, and vaginal vault prolapse, muscle flap reinforcement, and reconstruction of the pelvic floor and sacrocolposuspension. This invention is thought to be particularly well-suited for use in low-pressure procedures where the overall level of stress generated in the implant member 1 is not high.

With continued reference now to FIG. 1, the implant member 1 has a length L in the direction of axis Z, width W in the direction of axis Y, and thickness T in the direction of axis X.

The thickness T is of particular importance because it is one of the factors that affects how the implant member 1 “handles”; a thin piece of material will be more supple than a thicker piece of material, and so the thin piece of material can better conform to the patient's anatomy. However, because the ability of the material to support tensile loads depends, in part, upon the material's thickness, a thin piece of material may not be strong enough to support all loads applied. Accordingly, the thickness of the material should be chosen so that the material will be sufficiently flexible, yet also will be strong enough to support all of the forces that it may be subjected to when implanted in the body.

By way of non-limiting example, the preferred thickness T of the implant member 1 is about 0.8-1.5 mm; thinner material can be used but, depending upon the load applied, it may deform excessively or even fail. Consequently, material thinner than about 0.8 mm preferably will not be used in most circumstances. Thicker material also can be used, although it should be understood that material greater than 1.5 mm may be too thick because it might be noticeable to the patient, and also might be so stiff that it could be difficult for the surgeon to work with, so such thicker material also will not be used in most circumstances.

The length L of the implant member 1, which is intended to be used as a patch or support, is preferably between 7-8 cm, and the width is preferably between 4-6 cm. These dimensions have been chosen because surgeons already use patches of other materials made in these sizes for treatment such as prolapse repair; accordingly, it should be understood that these dimensions are provided by way of non-limiting example only. Larger or smaller patches, and patches having different length:width ratios could be used, without departing from this invention.

It also will be appreciated that the implant member 1 could be trimmed as needed prior to use, whether because of the patient's anatomy or because less than the full amount of the implant member is needed.

With continued reference to FIG. 1, the slits 3 formed in the implant member 1 are preferably arranged in a regular and repeating pattern. By way of non-limiting example, the slits can be approximately 3.7 mm in length. The length and width of each slit 3 will depend upon the way that the slit 3 is formed.

As can be seen in FIG. 1, the slits 3 in the implant member 1 are formed in rows that run along the length of the implant member 1 in lines parallel to axis Z. Slits are arranged in a “row” where those slits are all line segments which are lie on a single line. The slits 3 are preferably arranged in a staggered fashion; as shown in FIG. 1, alternating rows of slits 3A and 3B are placed so that, moving in the widthwise direction along axis Y, slits in rows 3A do not lie directly adjacent to and in registry with the slits in rows 3B. Instead, moving widthwise along axis Y from a slit in any given row 3A one then encounters the solid material between the slits in the adjoining row 3B and then the slit in the row 3A that follows the row 3B. This is done in order to distribute better the tensile forces that are applied to implant member 1.

Alternatively, slits 3 can be arranged so that the slits 3 in alternating (rather than adjacent) rows 3A and 3B are disposed in registry (not shown).

“Staggered” also can be construed more broadly to mean that the rows are arranged in any manner such that a slit in one row does not lie directly alongside and in registry with a slit in an adjacent row. “Staggered” would, therefore, encompass arrangements where there is partial overlap of slits 3 in adjacent rows (not shown).

The arrangement and quantity of slits 3 will affect the properties of the implant member 1. As the number and/or length of the slits increases, the implant member 1 will stretch more under a given load. An implant member 1 having a large number of slits will be more pliable than a member having a lower number of slits, but it may not be as strong. The number and arrangement of slits can, therefore, be chosen to provide an implant member 1 with the appropriate levels of strength and flexibility.

So too, slit size can be varied to control the elastic properties of the implant member 1. As larger slits 3 are formed, the implant member 1 will stretch more under a given load, and so will not be able to as large a maximum load before failing.

It also should be understood that the slits could be arranged to lie parallel to the direction in which force is applied to the implant member (not shown). In that case, the applied force will not cause the slits to open; however, bending or twisting of the support member as it conforms to the internal body structure may cause some slits to open.

The slits can be formed in the suitable source material using a skin graft mesher. Skin graft meshers are known and are currently used in connection with the treatment of burns. These devices allow a skin graft of a particular size to be expanded so as to cover a greater area wound. Skin graft meshers are described in U.S. Pat. No. 5,004,468, No. 5,219,352 and No. 5,306,279, all assigned to Zimmer, Inc., of Warsaw Ind., and No. 6,063,094, assigned to L.R. Surgical Instruments Ltd. of Ofakim, Israel. These devices use one or more bladed cylindrical cutters and support carrier to produce an array of slits in the skin graft. The meshing ratio, also known as a slit ratio, (i.e., 1.5:1, 3:1 or 6:1) refers to the approximate amount by which the graft expands; for example, a 1.5:1 meshing ratio provides a graft that covers approximately 1.5 times the area of the original graft. Different cutters are used to produce different mesh ratios. In general, as the mesh ratio increases, so does the number (or length) of slits that are formed in the graft.

Presently, a Zimmer Skin Graft Mesher is preferred. This device is manufactured by Zimmer, Inc., identified previously.

The present invention encompasses the use of slit ratios up to approximately 6:1.

A slit ratio of 1.5:1 is presently preferred because it results in an implant member 1 having both good strength and extensibility. As noted above, the slit ratio refers to the approximate amount by which the area of the resulting meshed graft is increased. A 1.5:1 ratio graft therefore will cover approximately 150% of the area of the source graft prior to meshing.

Ratios of 3:1 and 6:1 also could be used in this invention, depending upon the amount of force that will be applied to the implant member 1. These ratios are preferably produced with skin graft meshers, and it is noted that skin graft meshers come with cutters that can manufacture workpieces with such slit ratios. Other ratios may be produced by using meshers having custom cutters designed for a particular application.

In deciding which slit ratio to use, it should be understood that higher slit ratios, while they allow the use of less material and result in a more elastic implant member, may produce an implant member that can have difficulty supporting the maximum loads likely to be encountered when in the body.

Alternatively, the slits could be formed using a suitable die, or even by hand-slitting the source material with a blade. Other cutting techniques, such as water jet or laser beam, also could be used.

As an alternative to slits, holes could be formed in the implant member 1. Holes may enhance wound drainage (and so reduce wound dehiscence), but the elastic properties of the resulting implant member would not be the same. Also, unlike slits, where virtually no material is removed from the implant member 1, to form holes it is necessary to remove (and so waste) material from the implant member, since the holes must be formed by punching the implant member with a dies or cutter.

With reference now to FIG. 2, the depicted implant member 1, which includes an array of slits 3, is subjected to tension by force applied in the direction of arrow F. The applied force, which is preferably spread over the ends of the implant member 1 in generally uniform fashion so as to avoid stress concentrations that could damage or even tear the implant member 1, causes the slits 3 to open. The open slits 3 result in expansion of the implant member 1 proportionate to the magnitude of the applied force, upon to a maximum of approximately the implant member's slit ratio.

While the implant member 1 is under tension, the slits 3 define openings 5. Openings 5 provide at least two benefits. First, some of the patient's tissue may extend into at least some of the openings 5. Such ingrowth differs from ingrowth into the microstructure of the implant member 1; here, tissue will actually enter into and grow through the open slits 3 of the implant member (which is not to say that tissue also cannot grow into the microstructure of the implant member). Second, fluid exchange through the implant is enhanced, since fluid and suspended and dissolved materials can pass through the openings 5.

Should the implant member 1 be placed into the body without tension, slits 3 will allow the implant member 1 to conform more closely to the body's internal structure, and also to accommodate body movements. Additionally, tissue ingrowth through the slits 3 still can take place.

The precise shape of the openings 5 when the implant member 1 is placed under tension will be affected by both the length of the associated slit 3 and the direction and magnitude of the force that is applied. Viewed along axis X (looking in the direction perpendicular to the Y-Z plane) of FIGS. 1 and 2, when tension is applied along axis Y in a direction perpendicular to the rows 3A, 3B of slits 3, the openings 5 are approximately lens-shaped.

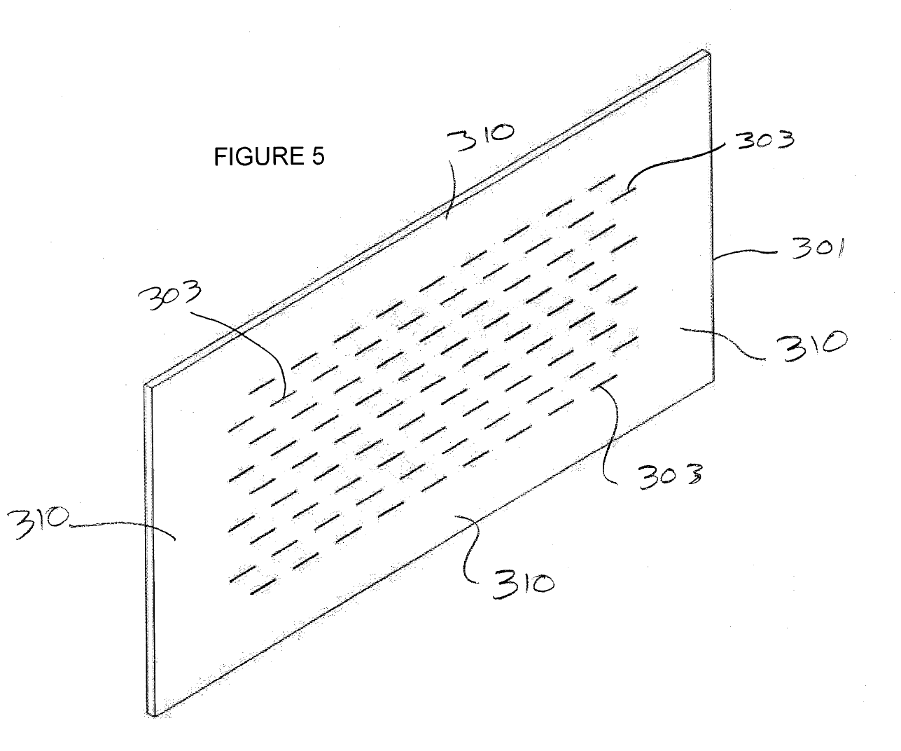

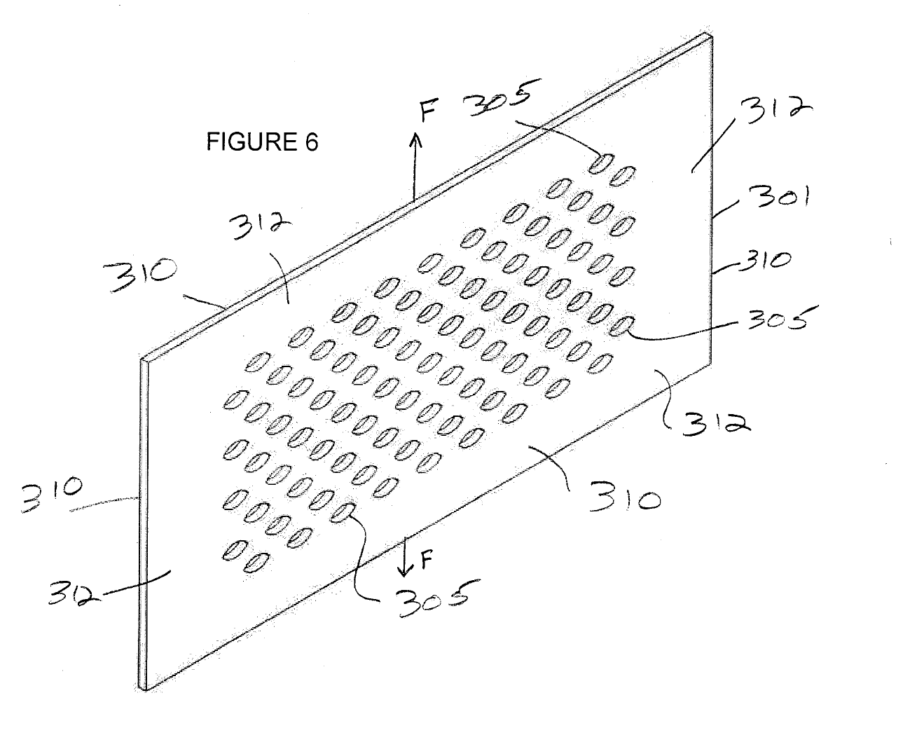

Optionally, as shown in FIGS. 5 and 6, the slits 303 can be eliminated at the edges 310 of the implant member so that the implant member 301 has a solid perimeter formed from solid regions 312. In this arrangement, the perimeter of the implant member 301 only can stretch to the extent permitted by the inherent elasticity of the material from which the implant member 301 is made. The inner portion of the implant member 301 has slits 303, and so still can deform in response to the application of force F by forming openings 305 as discussed above, and depicted in FIG. 6.

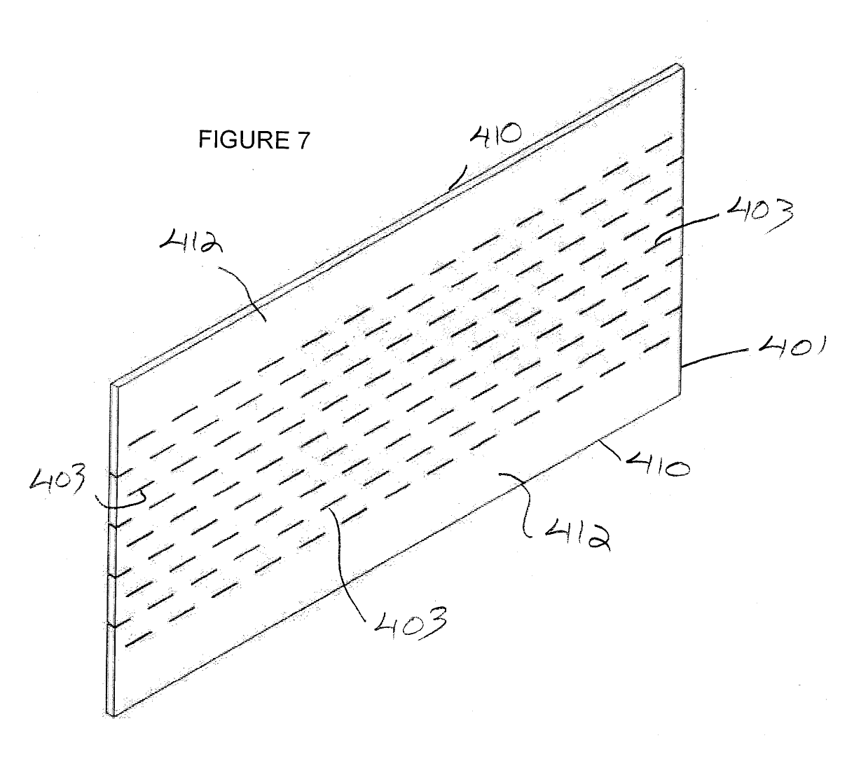

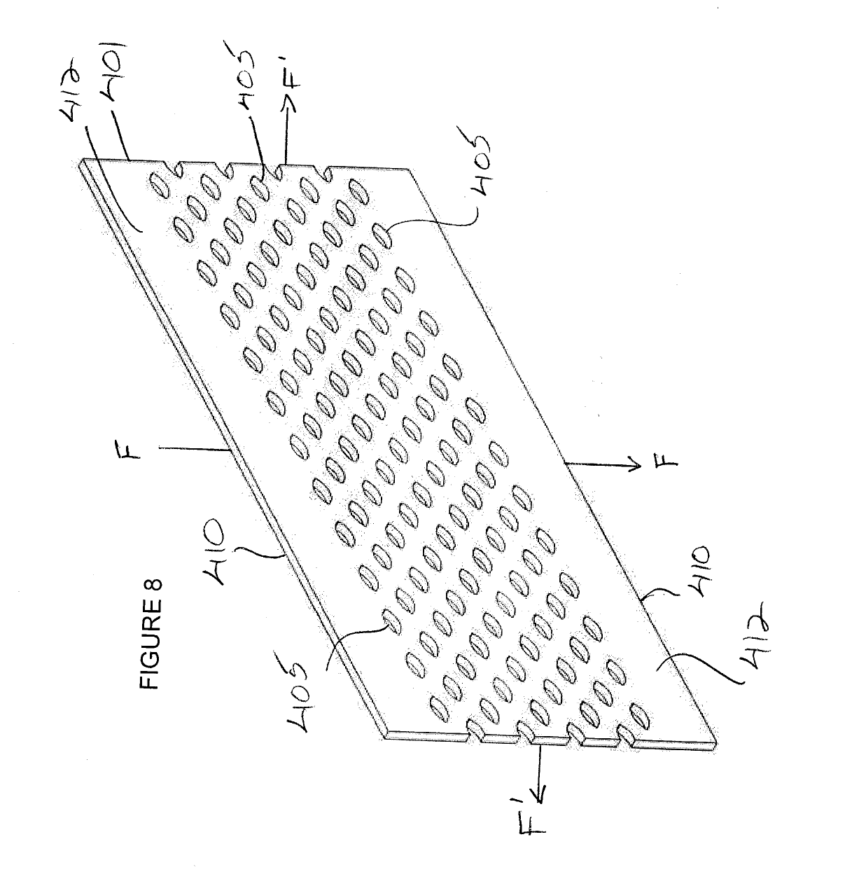

Also optionally, as shown in FIGS. 7 and 8, the slits 403 can be eliminated at just two of the edges 410 of the implant member so that the implant member 401 has two solid perimeter regions 412. In this arrangement, the perimeter of the implant member 401 only can stretch to the extent permitted by the inherent elasticity of the material from which the implant member 401 is made, whereas the inner portion having the slits 403 can deform to a greater extent, as discussed above, and depicted in FIG. 8. In FIG. 8 tension is applied in the direction of arrows F; however, it will be understood that there may be situations where it is preferable to apply force in the same direction as the lines on which slits 403 are arranged (arrows F′).

It also should be understood that the implant member 1 could be provided with at least one section where no slits are formed. This will alter the elastic properties of the implant member. By way of non-limiting example, the implant strip could have two rectangular regions running parallel to the length of the implant strip, that is, in the direction of axis Z. These rectangular regions could be symmetrically arranged about the centerline of the implant strip 1.

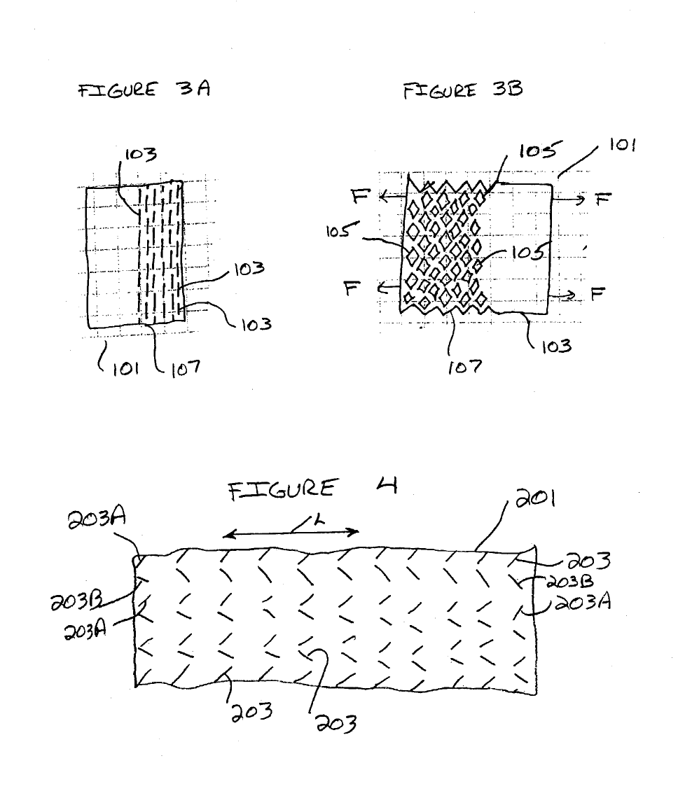

FIGS. 3A and 3B depict deformation of an implant member 101 in which a portion of the implant member 101 does not have slits 103 in response to applied force exerted along the length of the implant member 101.

FIG. 3A shows the implant member 101, including slits 103, in the relaxed state. Owing to the inherent elasticity of the material from which implant member 101 is made, the slits 103 remain closed.

FIG. 3B shows the implant member 101 subjected to tensile force F applied along the length of the implant member 1, in a direction perpendicular to the rows of the slits. Such force F could be applied to each end of the implant member 101 over an area or at one or more discrete points; uniform loading is preferred as it avoids stress concentrations that could damage the implant member material. The difference in shape between the unloaded and loaded implant member 101 can be seen by comparing FIGS. 3A and 3B.

The tensile force F causes the slits 103 to deform and change shape to openings 105, which are approximately lens-shaped. Again, the precise shape of the openings 105 will depend upon the size and spacing of the slits 103 and the properties of the material from which the implant member 101 is made. As the tensile force increases, the openings 105 may become more diamond-shaped, as shown in FIG. 3B.

The implant member 101 is preferably made from material which retains its elasticity, and so, when tension is not applied to the implant member 101, the inherent resiliency of the material closes slits 103.

The slits 103 can be distributed uniformly and in parallel, as shown in FIGS. 1 and 2. Alternatively, the slits 103 could be distributed in an asymmetric manner (not shown). For example, the implant member 101 can be formed with fewer slits 103 near its perimeter, and more slits near its center. This will maintain strength and reduce elastic deformation at the perimeter of the implant member 107.

Although the foregoing embodiments of this invention preferably employ acellular porcine dermal tissue, this invention is not to be limited thereto. Any other suitable material, whether natural or synthetic, or even a combination thereof, can be used. Other examples of suitable materials that could be used with this invention include allografts, xenografts and autografts, and absorbable and non-absorbable synthetic materials.

Although FIGS. 1 and 2 depict an implant member 1 in which slits 3 are formed in lines parallel to the long axis of the implant member, this invention is not limited to those arrangements. By way of non-limiting examples, all of the slits could be formed, parallel to one another, at any angle between 0-180° to the implant member's long axis.

Nor must all of the slits be arranged in parallel to each other. With reference now to FIG. 4, and by way of non-limiting example, an implant member 201 can be constructed having rows of slits 203A oriented at a first angle and alternating with other rows of slits 203B oriented at a second angle relative to the long axis of the implant member 201. This results in a “herringbone” pattern of slits. It will be further appreciated that force could be applied either along or at right angles to the long axis of the implant member 201, shown as arrow L. Further, there may be other situations where it is desirable to apply force to the implant member 201 at some other angle. In that case, owing to the different orientations of the slits in rows 203A and 203B, the implant member 201 may have different tensile properties along its length and width

As a further variation, slits intersecting at right angles to form “+”-shaped slits could be arranged in a grid pattern. As a still further variation, in order to increase isotropy of the implant member a second grid of “+”-shaped slits, rotated by 45°, could then be interlaced with the first grid of slits. Other arrangements of “+”-shaped slits, or other shapes of intersecting slits, also could be used. Such slits could be formed in a single pass using correspondingly-shaped skin graft mesher cutters or in multiple passes, with slits of one orientation being formed in one pass, slits in another orientation being formed in a different pass. Such slits also could be formed using other techniques, such as blades or dies.

Another way to obtain an implant member with more uniform tensile properties would be to form the slits in the implant member with a random arrangement. Since the slits as a group are arranged without any particular preferred direction, the resulting implant member should not elongate in any one direction more than another (this presumes the number of slits is sufficient to offset the effect of any one slit).

Also by way of example only and not limitation, one side of the implant member could be formed with more or larger slits than the other in order to provide asymmetrical elastic properties (not shown). When placed in the patient's body, the more heavily perforated portion of the implant member will expand to a greater degree than the other portion of the implant member.

It is envisioned that this invention will be used in low-tension and low-tissue pressure tissue restoration operations, such as rectocele, cystocele and enterocele repairs. Vaginal vault prolapse and abdominal sacrocolpopexies and pelvic floor reconstructions also could be treated.

If this invention is to be used in higher-pressure applications, then the dimensions and/or properties of the implant material can be altered to compensate for the higher stress levels that will be encountered.

Thus, while there have been shown and described and pointed out novel features of the present invention as applied to preferred embodiments thereof, it will be understood that various omissions and substitutions and changes in the form and details of the disclosed invention may be made by those skilled in the art without departing from the spirit of the invention. It is the intention, therefore, to be limited only as indicated by the scope of the claims appended hereto.

It is also to be understood that the following claims are intended to cover all of the generic and specific features of the invention herein described and all statements of the scope of the invention which, as a matter of language, might be said to fall therebetween.

What is claimed is:

1. An implant member comprising a body made from a biocompatible material and having a plurality of slits formed therein, the slits opening when a tensile force is applied to the body.

2. An implant member according to claim 1, wherein the slits are arranged in a plurality of rows.

3. An implant member according to claim 2, wherein at least some of the rows are parallel to each other.

4. An implant member according to claim 1, wherein the body has a lengthwise axis and the slits are arranged parallel to the lengthwise axis.

5. An implant member according to claim 1, wherein the body has a lengthwise axis and the slits are arranged perpendicular to the lengthwise axis.

6. An implant member according to claim 1, wherein the slits are arranged in a first row and a second row, and the slits in the first row are staggered in position relative to the slits in the second row.

7. An implant member according to claim 6, wherein the first row is adjacent to the second row.

8. An implant member according to claim 6, wherein the slits in said first row are uniformly spaced and the slits in the second row are uniformly spaced and arranged so that the slits in the first row do not lie directly adjacent to and in registry with the slits in the second row.

9. An implant member according to claim 1, wherein at least some of the slits are arranged in an asymmetric manner so that they are not parallel to each other.

10. An implant member according to claim 1, wherein the slits are formed so that the implant member has a slit ratio of approximately 1.5:1.

11. An implant member according to claim 1, wherein the slits are formed so that the implant member has a slit ratio of approximately 3:1.

12. An implant member according to claim 1, wherein the slits are formed so that the implant member has a slit ratio of approximately 6:1.

13. An implant member according to claim 1, wherein the slits are formed so that the implant member has a slit ratio of not more than 6:1.

14. An implant member according to claim 1, wherein the body comprises natural material.

15. An implant member according to claim 1, wherein the body comprises acellular porcine dermal tissue.

16. A method of manufacturing an implant member, comprising the steps of:
providing a body; and
forming a plurality of slits in the body, the slits being dimensioned and disposed so that the slits open when a tensile force is applied to the body.

17. A method according to claim 16, wherein the slits are arranged in a plurality of rows.

18. A method according to claim 16, wherein at least some of the rows are parallel to each other.

19. A method according to claim 16 wherein the body has a lengthwise axis and the slits are arranged parallel to the lengthwise axis.

20. A method according to claim 16, wherein the body has a lengthwise axis and the slits are arranged perpendicular to the lengthwise axis.

21. A method according to claim 16, wherein the step of forming the slits comprises using a skin graft mesher to create the slits in the body.

22. A method according to claim 16, wherein the slits are arranged in a plurality of rows, and the slits in each row are staggered in position relative to the slits in an adjacent said row.

23. A method according to claim 22, wherein the slits in a first said row are uniformly spaced and the slits in a second said row that is adjacent to the first said row are uniformly spaced and arranged so that the slits in the second said row do not lie directly adjacent to and in registry with the slits in the second said row.

24. A method according to claim 16, wherein the slits are formed so that the body has a slit ratio of approximately 1.5:1.

25. A method according to claim 16, wherein the slits are formed so that the implant member has a slit ratio of approximately 3:1.

26. A method according to claim 16, wherein the slits are formed so that the implant member has a slit ratio of approximately 6:1.

27. A method according to claim 16, wherein the slits are formed so that the implant member has a slit ratio of not more than 6:1.

28. A method according to claim 16, wherein the body comprises natural material.

29. A method according to claim 16, wherein the body comprises acellular porcine dermal tissue.
